(19) United States
(12) Patent Application Publication
(10) Pub. No.: US 2018/0136181 A1
(43) Pub. Date: May 17, 2018

(54) COMPOSITE FILLER STRUCTURE, ELECTRONIC DEVICE INCLUDING THE SAME, AND METHOD OF MANUFACTURING THE SAME

(71) Applicant: Samsung Electronics Co., Ltd., Suwon-si (KR)

(72) Inventors: Minjong Bae, Yongin-si (KR); Jinhong Kim, Seoul (KR); Hajin Kim, Hwaseong-si (KR); Haengdeog Koh, Hwaseong-si (KR); Doyoon Kim, Hwaseong-si (KR); Seyun Kim, Seoul (KR); Soichiro MIZUSAKI, Suwon-si (KR); Hiesang Sohn, Seoul (KR); Changsoo Lee, Seoul (KR)

(21) Appl. No.: 15/810,229

(22) Filed: Nov. 13, 2017

(30) Foreign Application Priority Data
Nov. 15, 2016 (KR) 10-2016-0152242
Oct. 17, 2017 (KR) 10-2017-0134819

(51) Int. Cl. G01N 33/00; H01B 1/14; H01B 5/14

(52) U.S. Cl. CPC G01N 33/0027; H01B 5/14; H01B 1/14

(57) ABSTRACT

A composite filler structure includes a substrate, a filler layer spaced apart from the substrate and comprising a matrix material layer and a plurality of conductive filler particles, an electrode in contact with the filler layer and configured to provide an electrical signal to the filler layer, and an insulating layer between the substrate and the electrode, and including an alkali oxide in an amount of about 7 weight percent or less, based on a total weight of the composite filler structure.

### CROSS-REFERENCE TO RELATED APPLICATIONS

This application claims priority to and the benefit of Korean Patent Application No. 10-2016-0152242, filed on Nov. 15, 2016, and Korean Patent Application No. 10-2017-0134819, filed on Oct. 17, 2017, in the Korean Intellectual Property Office, and all the benefits accruing therefrom under 35 U.S.C. § 119, the contents of which in their entirety are herein incorporated by reference.

### BACKGROUND

### 1. Field

The present disclosure relates to a composite filler structure, an electronic device including the same, and a method of manufacturing the composite filler structure.

### 2. Description of the Related Art

A composite material is a material formed by combining two or more different materials having different physical and/or chemical properties, and has properties which are different from each single material making up the composite. For example, a composite material may be composed of a matrix providing a basic framework and a reinforcing material, such as a filler, to improve the properties thereof. Also, the composite material may be applied to various fields according to the component materials.

The composite material may be used as a conductive material or as an insulating material according to a content rate of a conductive filler.

### SUMMARY

Provided are composite filler structures and electronic devices including the same.

Provided are composite filler structures with enhanced withstanding voltage properties at a high temperature and electronic devices including the same.

According to an aspect of an embodiment, a composite filler structure includes, a substrate; a filler layer spaced apart from the substrate and comprising a matrix material layer and a plurality of conductive filler particles; an electrode in contact with the filler layer, and configured to provide an electrical signal to the filler layer; and an insulating layer between the substrate and the electrode, the insulating layer including an alkali oxide in an amount of about 7 weight percent (wt %) or less.

The insulating layer, the matrix material layer, or a combination thereof may include a glass frit.

The glass frit may include an oxide glass frit.

A glass transition temperature of the insulating layer may be about 400° C. or greater.

The composite filler structure may further include a protection layer in contact with the substrate and configured to prevent oxidation of the substrate.

The insulating layer may include a material which is the same as a material included in the protection layer.

Materials included in the insulating layer and the protection layer may be included in different amounts. In an embodiment, an amount of the same material in the insulating layer is different from an amount in the protection layer

A coefficient of thermal expansion of the insulating layer may range from about 5 micrometers per meter per degree (μm/m/deg) to about 12 μm/m/deg.

The electrode may be between the filler layer and the substrate.

The filler layer may be between the electrode and the substrate.

The filler layer may include a material configured to generate heat in response to an electrical signal.

The filler layer may include a material having a resistance which changes when contacted by gas particles.

The filler layer may include carbon black, graphite, a metal, a conductive polymer, a metal powder, a carbon nanotube (CNT), an oxide, a boride, a carbide, a chalcogenide, or a combination thereof.

According to an aspect of another embodiment, an electronic device includes a composite filler structure including: a substrate; a filler layer spaced apart from the substrate and including a matrix material layer and a plurality of conductive filler particles; an electrode in contact with the filler layer and configured to provide an electrical signal to the filler layer; and an insulating layer between the substrate and the electrode, the insulating layer including an alkali oxide in an amount of about 7 weight percent (wt %) or less.

The insulating layer, and the matrix material layer, or a combination thereof, may include a glass frit.

The substrate may include a cavity defined therein.

The composite filler structure may further include a protection layer in contact with the substrate and configured to prevent oxidation of the substrate.

The amount of alkali oxide in the insulating layer may be less than an amount of alkali oxide in the protection layer.

The filler layer may be configured to emit light by generating heat.

The electronic device further may include a gas chamber including a gas inlet through which gas is introduced into the gas chamber, and a photodetector configured to detect light transmitted by the filler layer and through the gas chamber.

According to an aspect of another embodiment, an insulating substrate includes a base layer including an electrically conductive material; and an insulating layer disposed on the base layer, the insulating layer including an alkali oxide in an amount of about 7 wt % or less, based on a total weight of the insulating layer.

According to an aspect of yet another embodiment, disclosed is a method of manufacturing a composite filler structure, the method including: providing a substrate; providing a filler layer spaced apart from the substrate and comprising a matrix material layer and a conductive filler; disposing an electrode in contact with the filler layer and configured to provide an electrical signal to the filler layer; and providing an insulating layer between the substrate and the electrode, the insulating layer comprising an alkali oxide in an amount of about 7 weight percent or less, based on a total weight of the composite filler structure.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the example embodiments, taken in conjunction with the accompanying drawings in which:

FIG. 1 is a cross-sectional view of an embodiment of a composite filler structure;

FIG. 2 is a plan view of the composite filler structure of FIG. 1;

FIG. 3A is a table showing the composition of a glass frit material included in a protection layer of a composite filler structure according to an example embodiment;

FIG. 3B is a table showing the results of measuring a withstanding voltage of a composite filler structure in which an insulating layer includes the same material as that of a protection layer, according to an example embodiment;

FIG. 4A is a table showing the composition of an insulating layer in which the amount of alkali oxide is less than the composition shown in FIG. 3A according to an example embodiment;

FIG. 4B is a table showing the results of measuring a withstanding voltage of a composite filler structure including the insulating layer having the composition in FIG. 4A according to an example embodiment;

FIG. 5 is a cross-sectional view of a composite filler structure according to another example embodiment;

FIG. 6 is a cross-sectional view of a composite filler structure in which an electrode is disposed on an upper surface of a filler layer according to an example embodiment;

FIG. 7 is a cross-sectional view of a composite filler structure in which first and second electrodes are disposed with a filler layer therebetween according to an example embodiment;

FIG. 8A is an illustration of a heating device including a composite filler structure according to an example embodiment;

FIG. 8B is a cross-sectional view of a composite filler structure present in the circled portion of FIG. 8A;

FIG. 9 illustrates an example of a gas sensor including a composite filler structure; and

FIG. 10 is a cross-sectional view of an example of a substrate having an insulating property.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. Like reference numerals refer to like elements throughout. As used herein, the term “and/or” includes any and all combinations of one or more of the associated listed items. “Or” means “and/or.” Expressions such as “at least one of,” when preceding a list of elements, modify the entire list of elements and do not modify the individual elements of the list.

It will be understood that when an element is referred to as being “on” another element, it can be directly on the other element or intervening elements may be present therebetween. In contrast, when an element is referred to as being “directly on” another element, there are no intervening elements present.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third,” etc. may be used herein to describe various elements, components, regions, layers and/or sections, these elements, components, regions, layers and/or sections should not be limited by these terms. These terms are only used to distinguish one element, component, region, layer or section from another element, component, region, layer, or section. Thus, “a first element,” “component,” “region,” “layer,” or “section” discussed below could be termed a second element, component, region, layer or section without departing from the teachings herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “At least one” is not to be construed as limiting “a” or “an.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

As used herein, “composite” refers to a material formed by combining two or more materials having different physical and/or chemical properties, wherein the composite has properties different from each material constituting the composite individually, and wherein particles or wires of each material are at least microscopically separated and distinguishable from each other in a finished structure of the composite.

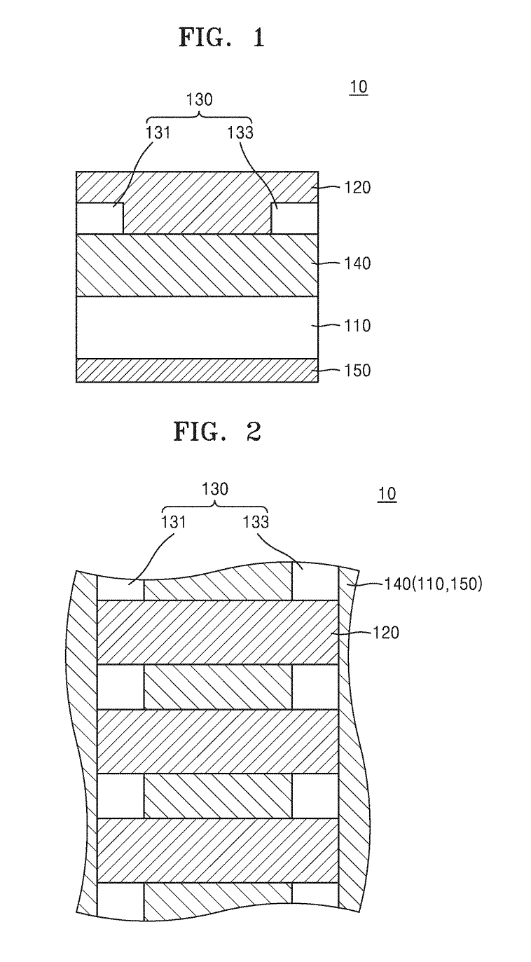

FIG. 1 is a cross-sectional view of a composite filler structure 10 according to an example embodiment. FIG. 2 is a plan view of the composite filler structure 10 of FIG. 1.

As shown in FIGS. 1 and 2, the composite filler structure 10 may include a substrate 110 having electrical conductivity, a filler layer 120 spaced apart from the substrate 110 and including a plurality of conductive filler particles, electrodes 130 contacting the filler layer 120 and providing an electrical signal to the filler layer 120, and an insulating layer 140 disposed between the substrate 110 and the electrodes 130 and preventing the generation of a leakage current between the substrate 110 and the electrodes 130. The composite filler structure 10 may further include a protection layer 150 in contact with the substrate 10 and configured to prevent oxidation of the substrate 10 (e.g., prevent the substrate 10 from being oxidized).

The substrate 110 may be a layer supporting the composite filler structure 10. The substrate 110 may include a material having relatively high mechanical strength. For example, the material may be a conductive metallic material, etc., having high mechanical strength. Thus, the substrate 110 may include a conductive material. For example, the substrate 110 may include a steel plate, for example, a steel plate porcelain (SPP) material. Alternatively, the substrate 110 may include a material such as iron (Fe), aluminum (Al), magnesium (Mg), titanium (Ti), zirconium (Zr), zinc (Zn), niobium (Nb), silver (Ag), gold (Au), copper (Cu), an alloy thereof, or a combination thereof, but is not limited thereto. The substrate 110 may be formed of an insulating material.

The filler layer 20 may include a matrix material layer and a plurality of filler particles having electrical conductivity. The filler layer 120 may generate heat in response to an electrical signal or may have an electrical conductivity, for example, an electrical resistance which changes when the material comes into contact with particles introduced to the filler layer from outside of the composite filler structure. The electrical conductivity, or the amount of heat generated by the filler layer 120, may be selected by controlling the amount of the filler particles in the filler layer 120. For example, as the amount of a plurality of filler particles in the filler layer 120 increases, the electrical conductivity of the filler layer 120 may also increase. An electrical signal flowing through the filler layer 120 may vary according to a change in the electrical conductivity.

The matrix material layer may include a glass material. For example, according to an example embodiment, the matrix material layer may include a glass frit or enamel powder (e.g., glass powder). As used herein, a “glass frit” refers to granular, crushed glass.

The plurality of filler particles can comprise different materials and/or types of particles. At least a portion of the plurality of filler particles may include a crystalline material. The plurality of filler particles may include a one-dimensional (1D) filler, a two-dimensional (2D) filler, or a combination thereof. In other words, the plurality of filler particles may include a nanostructure having a 1D or 2D extended structure. As used herein, the term “nanostructure” refers to a material having a least one dimension (e.g., a diameter or a thickness) which is on a nanoscale level, i.e., a dimension of less than about 1000 nanometers, or about 1 nm to about 1000 nm, about 2 nm to about 800 nm, or about 4 nm to about 600 nm. For example, according to an example embodiment, at least one of the plurality of filler particles may have a nano-wire shape, nano-rod shape, or a similar shape. As used herein, the term “nano-rod” refers to a material having a rod shape and which has at least one dimension (e.g. a diameter) in a range of less than about 1000 nanometers, or about 1 nm to about 1000 nm, and an aspect ratio of greater than or equal to 2. As used herein, the term “nano-wire” refers to a wire-like material which has a diameter on a nanoscale level, which is not limited by its length, and which has an aspect ratio of about 100 or more. The plurality of filler particles may not have a specific directionality or orientation and may be randomly arranged. In other words, the plurality of filler particles may be arranged in random directions. An improvement in the properties of the composite material may be determined based upon the type of filler particles included in the composite filler structure.

Alternatively, at least one of the plurality of filler particles may have a nano-sheet shape.

The plurality of filler particles may have a composition having an electrical conductivity of greater than about 750 Siemens per meter (S/m), or greater than about 1,000 S/m, or greater than about 1,100 S/m, as measured at room temperature (i.e., about 25° C.). In an embodiment, the filler has an electrical conductivity from about 750 S/m to about 2,000 S/m, or from about 800 S/m to about 1,750 S/m, or from about 1,000 S/m to about 1,500 S/m, or from about 1,000 S/m to about 1,250 S/m. In an embodiment, the filler has an electrical conductivity of about 1,250 S/m. However, the electrical conductivity may be less or greater depending on the case.

At least some of the plurality of filler particles may be in contact with each other. For example, among the plurality of filler particles, horizontally or vertically adjacent fillers may be in contact with each other. The plurality of filler particles are relatively uniformly distributed in the matrix material layer and may be electrically connected to each other. The plurality of filler particles may configure a network structure.

The plurality of filler particles may include an electrically conductive material. The plurality of filler particles may include a material that generates heat in response to an electrical signal. Alternatively, the plurality of filler particles may include a material having an electrical conductivity, for example, electrical resistance, which changes when contacted by particles introduced from outside of the composite filler structure. For example, the plurality of filler particles may include carbon black, graphite, a metal, a conductive polymer, a metal powder, a carbon nanotube (CNT), or a combination thereof, but is not limited thereto. Alternatively, the plurality of filler particles may include an oxide, a boride, a carbide, and a chalcogenide. The oxide may include, for example, RuO₂, MnO₂, ReO₂, VO₂, OsO₂, TaO₂, IrO₂, NbO₂, WO₂, GaO₂2, MoO₂, InO₂, CrO₂, RhO₂, or a combination thereof. The boride may include, for example, Ta₃B₄, Nb₃B₄, TaB, NbB, V₃B₄, VB, or a combination thereof. The carbide may include, for example, Dy₂C, Ho₂C, or a combination thereof. The chalcogenide may include, for example, AuTe₂, PdTe₂, PtTe₂, YTe₃, CuTe₂, NiTe₂, IrTe₂, PrTe₃, NdTe₃, SmTe₃, GdTe₃, TbTe₃, DyTe₃, HoTe₃, ErTe₃, CeTe₃, LaTe₃, TiSe₂, TiTe₂, ZrTe₂, HfTe₂, TaSe₂, TaTe₂, TiS₂, NbS₂, TaS₂, Hf₃Te₂, VSe₂, VTe₂, NbTe₂, LaTe₂, CeTe₂, or a combination thereof. A combination comprising at least one of the foregoing may also be used.

The dimension (e.g. a diameter or a thickness) of each particle of the plurality of filler particles may range from about 1 nanometer (nm) to about 1,000 nm, or from about 10 nm to about 900 nm, or from about 50 nm to about 750 nm, or from about 75 nm to about 500 nm. In an embodiment, a size of each of the plurality of filler particles may range from 100 nm to about 500 nm. An amount of each of the plurality of filler particles in the filler layer 120 may range from about 0.1 weight percent (wt %) to about 100 wt %, or from about 2 wt % to about 90 wt % or from about 10 wt % to about 75 wt %, based on a total weight of the filler layer.

The matrix material layer of the filler layer 120 may include a glass frit. The glass frit will be described later.

Alternatively, the matrix material layer may include a heat resistant organic material. For example, according to an embodiment, the matrix material layer may include an organic polymer, for example a thermoplastic polymer. The organic polymer may have a melting temperature of, for example, 200° C. or greater. The organic polymer may include polyimide (PI), polyphenylene sulfide (PPS), polybutylene terephthalate (PBT), polyamide imide (PAI), liquid crystalline polymer (LCP), polyethylene terephthalate (PET), polyphenylene sulfide (PPS), and polyetheretherketone (PEEK), or a combination thereof.

The filler layer 120 may be disposed on a surface of the substrate 110. For example, according to an embodiment, a region of the filler layer 120 overlapping the surface of the substrate 110 may be about 70% or more, or 80% or more, or 90% or more of the surface of the substrate 110, which may be referred to as planar type heating as heat may be generally generated on the substrate 110. The filler layer 120 may be formed as a single layer or as a plurality of filler layers.

A plurality of filler layers 120 may be arranged on the substrate 110. The plurality of filler layers 120 may be one-dimensionally or two-dimensionally spaced apart from one another on the substrate 110. In FIG. 2, the plurality of filler layers 120 are one-dimensionally arranged. Alternatively, at least two of the plurality of filler layers 120 may be connected to each other. The plurality of filler layers 120 have small spaces therebetween, thereby preventing the plurality of filler layers 120 from being bent due to thermal expansion, although the plurality of filler layers 120 expand due to heat.

Alternatively, a single filler layer 120 may be provided. For example, according to an example embodiment, the filler layer 120 may be arranged in a center region of the substrate 110. When only one filler layer 120 is provided, the filler layer 120 may include one or more openings. Thus, even though the filler layer 120 expands due to heat, a shape thereof may not be deformed.

The electrodes 130 may be adjacent to the filler layer 120. The electrodes 130 may include a first electrode 131 contacting one region of the filler layer 120 and a second electrode 133 contacting another region of the filler layer 120. A pair of electrodes 130 may be arranged for each filler layer 120. Thus, the electrodes 130 may provide an electrical signal to the filler layer 120, and the filler layer 120 may generate heat according to the electrical signal.

The electrodes 130 may include a material having excellent electrical conductivity. The first electrode 131 and the second electrode 133 may include Ag, Al, indium tin oxide (ITO), Cu, Mo, Pt, or a combination thereof, but are not limited thereto.

Meanwhile, the insulating layer 140 may be arranged between the electrodes 130 and the substrate 110 to prevent a leakage current from being generated between the substrate 110 and the electrodes 130 or between the substrate 110 and the filler layer 120. The insulating layer 140 may include a composition that is bonded (e.g., adhered) to the filler layer 120. The insulating layer 140 may include a glass frit. For example, the insulating layer 140 may include an oxide glass frit, including for example, B₂O₃, Na₂O, ZnO, Al₂O₃, BaO, P₂O₅, CuO, TiO₂, ZrO₂, BiO₂, NiO, CoO, or a combination thereof.

The insulating layer 140 according to an embodiment may include a glass frit. The insulating layer may not include an alkali oxide or may include a small amount of an alkali oxide, so as to have a high glass transition temperature with good adhesion to adjacent layers. For example, the insulating layer 140 may include an alkali oxide in an amount of about 7 wt % or less, or about 5 wt % or less, or about 1 wt % or less.

The insulating layer 140 may have a glass transition (Tg) temperature of about 400° C. or higher, or about 450° C. or higher, or about 500° C. or higher, or about 750° C. or higher. The glass transition temperature may be a value indicating heat resistance and may be measured by thermomechanical analysis (TMA), dynamic mechanical analysis (DMA), or the like. If the insulating layer 140 has a glass transition temperature Tg of 400° C. or higher, the insulating layer 140 may have excellent oxidation resistance and may efficiently block current flow even at a high temperature (e.g., 400° C. or higher), thereby providing a stable insulating property.

Alkali metal components (for example, Li+, Na+, K+) have a very small cationic radius and a low electrovalence. If the insulating layer 140 includes a large amount of alkali oxide, an excess of electrical conductive paths are generated. As a result, a discharge is generated in the insulating layer 140, and the material in the insulating layer 140 is damaged, thereby causing a thermal breakdown and a subsequent loss in the insulating property. A representative material where this phenomenon occurs may be enamel. Enamel contains an alkali metal in an amount of about 11 wt % or more, and thus as the temperature increases, a leakage current increases. As a result, the insulating property is lost at a temperature of about 200° C. or higher.

Thus, by including no alkali oxide or including a small amount of alkali oxide, the insulating layer 140 according to an embodiment may effectively block current flow even at high temperatures (for example, 400° C. or higher). That is, the insulating layer 140 may have excellent insulation properties and secure electrical stability. The alkali oxide may include Li₂O, Na₂O, K₂O, and the like. The insulating layer 140 may include an alkali oxide in an amount of about 7 wt % or less so as to maintain the insulating property at 400° C. or higher, but is not limited thereto. The amount of the alkali oxide varies depending on a desired temperature range of the highest tolerated temperature. That is, the insulating layer 140 may include the alkali oxide in an amount of 7 wt % or less in order to maintain the insulating property at a temperature of about 700° C. or higher.

The insulating layer 140 may have a thickness of about 100 micrometers (μm) to about 300 μm. The insulating layer 140 may have a thickness of, for example, about 100 μm to about 280 μm, for example, a thickness of about 100 μm to about 250 μm, for example, a thickness of about 100 μm to about 230 μm, and for example, a thickness of about 100 μm to about 180 μm. If the thickness of the insulating layer 140 is less than the above-described ranges, the insulating effect may be low and damage may be incurred in the event of an external impact. Conversely, if the thickness exceeds the above-described ranges, the cost may increase but the heating efficiency may be reduced, and thus the insulating layer 140 may be appropriately used within the above ranges. The insulating layer 140 may be a single layer or a plurality of layers.

The protection layer 150 covering the substrate 110 may be disposed in order to prevent the substrate 110 from being exposed to the outside (e.g., an environment external to the composite filler structure) and subsequently oxidized. The insulating layer 140 and the protection layer 150 may each include the glass material, for example, the glass frit, similar to the filler layer 120.

The coefficient of thermal expansion for each of the substrate 110, the filler layer 120, the insulating layer 140, and the protection layer 150 may range from about 5 micrometers per meter per degree (μm/m/deg) to about 12 μm /m/deg, or from about 7 μm/m/deg to about 12 μm/m/deg. Alternatively, coefficients of thermal expansion of the substrate 110, the filler layer 120, the insulating layer 140, and the protection layer 150 may range from about 9 μm/m/deg to about 11 μm/m/deg. The substrate 110, the filler layer 120, the insulating layer 140, and the protection layer 150 may have similar coefficients of thermal expansion. Without being limited by theory, if the coefficients of thermal expansion between the layers are similar, stress due to a thermal deformation may be small.

The substrate 110 may include the steel plate having a high mechanical strength in order to maintain an outer shape of a device, whereas the filler layer 120, the insulating layer 140, and the protection layer 150 may each include a glass frit in order to facilitate high levels of bonding with the substrate 110 or therebetween, and a similar coefficient of thermal expansion.

That is, each of the filler layer 120, the insulating layer 140, and the protection layer 150 may include the glass frit to increase adhesion therebetween. The glass frit may include an oxide glass frit. For example, the glass frit may include an oxide including silicon oxide, lithium oxide, nickel oxide, cobalt oxide, boron oxide, potassium oxide, aluminum oxide, titanium oxide, manganese oxide, copper oxide, zirconium oxide, phosphorus oxide, zinc oxide, bismuth oxide, argon oxide, lead oxide, sodium oxide, or a combination thereof.

The glass frit may include an oxide glass frit including an additive. The additive may include lithium (Li), nickel (Ni), cobalt (Co), boron (B), potassium (K), aluminum (Al), titanium (Ti), manganese (Mn), copper (Cu), zirconium (Zr), phosphorus (P), zinc (Zn), bismuth (Bi), lead (Pb), sodium (Na), or a combination thereof, but is not limited thereto.

At least some of the material included in the insulating layer 140 may be the same as the materials included in the protection layer 150. Alternatively, the protection layer 150 may include all of the same materials included in the insulating layer 140. In the insulating layer, an amount of the material may be different from an amount of the material included in the protection layer. Although the insulating layer 140 and the protection layer 150 include the same material, since the insulating layer 140 is designed to prevent the generation of a leakage current between the electrodes 130 and the substrate 110, and the protection layer 150 is designed to prevent oxidation of the substrate 110, an amount of the material in the insulating layer may be different from an amount of the material in the protection layer. For example, according to an example embodiment, an amount of alkali oxide included in the insulating layer 140 may be less than an amount of alkali oxide included in the protection layer 150. The content rate of alkali oxide included in the insulating layer 140 may be 7 wt % or less, or about 5 wt % or less, or about 1 wt % or less. The content rate of alkali oxide included in the protection layer 150 may be greater than about 7 wt %, or greater than 10 wt % or greater than 15 wt %. Alternatively, the insulating layer 140 may not include alkali oxide, and the protection layer 150 may include alkali oxide.

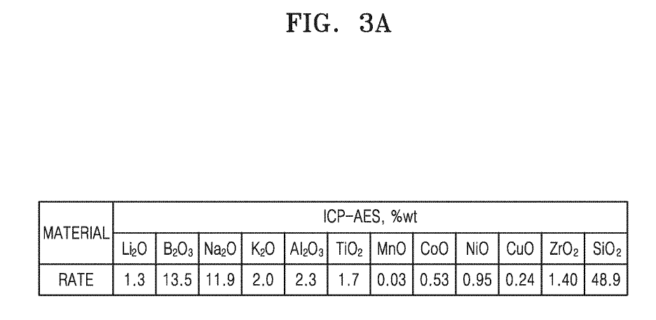

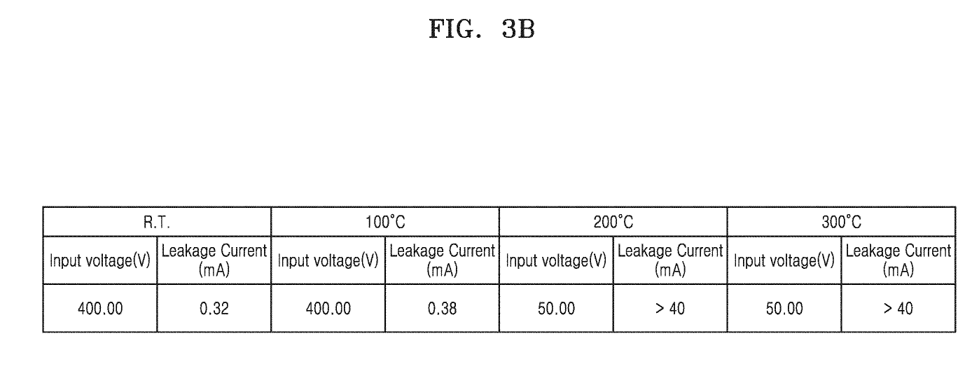

FIG. 3A is a table showing a composition of a glass frit material included in the protection layer of the composite filler structure of FIG. 1, according to an example embodiment. FIG. 3B is a table showing the result of measuring a withstanding voltage of a composite filler structure in which an insulating layer includes the same material as that of a protection layer, according to an example embodiment. As shown in FIG. 3A, the protection layer includes LiO, B₂O₃, Na₂O, K₂O, Al₂O₃, TiO₂, MnO, CoO, NiO, CuO, ZrO₂, and SiO₂. The insulating layer including the same material as that of the protection layer, has a thickness of about 150 μm, and is formed between a substrate and an electrode. A leakage current of the insulating layer is measured with respect to temperatures and input voltages. That is, the leakage current is measured with respect to various input voltages and temperatures, and the results are shown in FIG. 3B. As shown in FIG. 3B, the insulating layer generates a leakage current of about 40 milliamperes (mA) at a small input voltage of about 50 volts (V) at a temperature of about 200° C. Thus, the protection layer including LiO, B₂O₃, Na₂O, K₂O, Al₂O₃, TiO₂, MnO, CoO, NiO, CuO, ZrO₂, and SiO₂ has a low withstanding voltage at a high temperature.

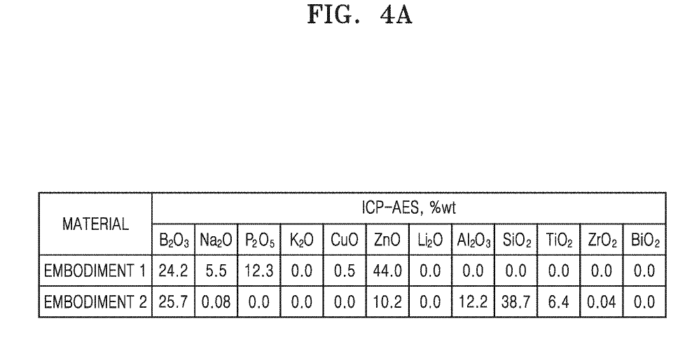

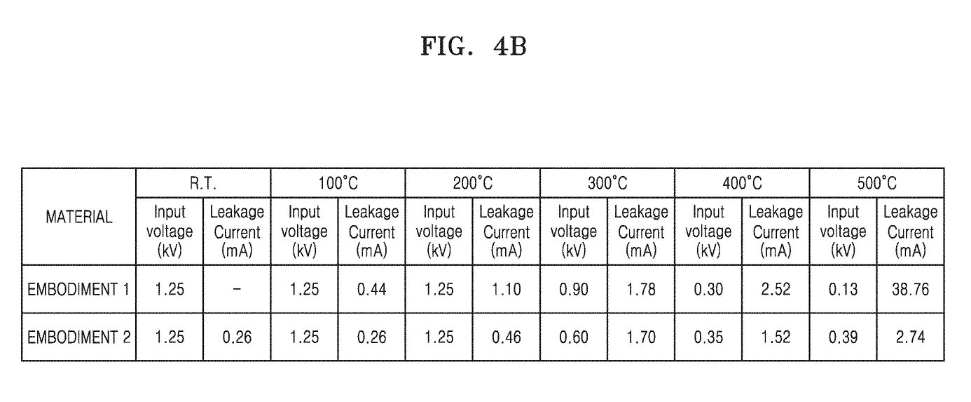

FIG. 4A is a table showing a composition of two insulating layers (Embodiment 1 and Embodiment 2) having a lower amount of alkali oxide lower than the insulating layer in FIG. 3A, according to an example embodiment. FIG. 4B is a table showing a result of measuring a withstanding voltage using the insulating layer of FIG. 4A, according to an example embodiment.

As shown in FIG. 4A, the insulating layers of Embodiments 1 and 2 do not include Li₂O and K₂O (e.g., no alkali oxide) or have a low amount of the alkali oxide Na₂O. In Embodiment 1, the amount of Na₂O may be about 5.5 wt %. In Embodiment 2, the amount of Na₂O may be about 0.08 wt %. Composite filler structures containing an insulating layer of Embodiment 1 or Embodiment 2, having the amounts of the alkali oxide material and a thickness of about 150 μm are formed, and then leakage currents thereof are measured at various temperatures and input voltages. As shown in FIG. 4B, at a high input voltage and a high temperature, the insulating layers generate no leakage current or only a minor amount of leakage current, and at a high temperature of about 500° C., generate a leakage current less than about 40 mA.

Depending upon a type and amount of a glass frit, the glass frit may only weakly prevent a leakage current from being generated or may not be effective at all to prevent a leakage current from being generated. Thus, the insulating layer may include a glass frit which is different from the glass frit included in the protection layer or may contain an amount of the glass frit which is different from an amount of glass frit in the protection layer, even if the insulating layer includes the same material as that of the protection layer. In particular, the insulating layer may contain a glass frit having a lower amount of alkali oxide than the protection layer, or the insulating layer may contain a glass frit having no alkali oxide, and thus a high temperature withstanding voltage property of a composite filler structure may be enhanced. If the insulating layer contains an amount of alkali oxide of about 7 wt % or less, the high temperature withstanding voltage property of a composite filler structure may be enhanced.

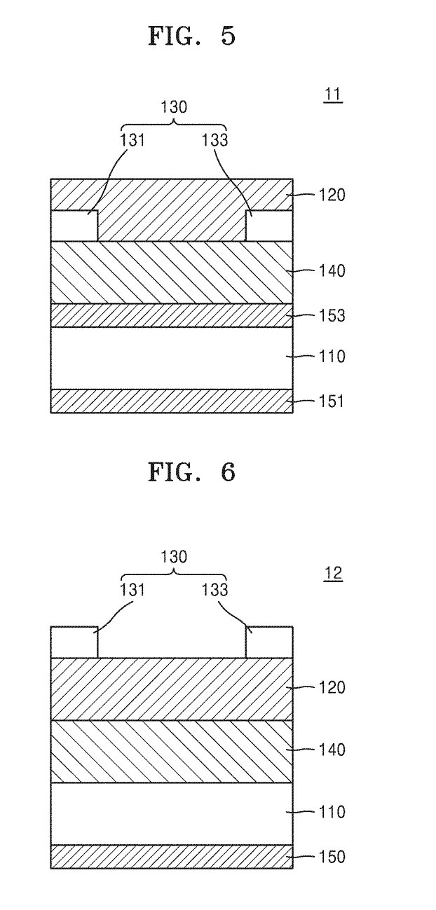

FIG. 5 is a cross-sectional view of a composite filler structure 11 according to another example embodiment. Upon comparing FIGS. 1 and 5, the composite filler structure 11 according of FIG. 5 may further include a protection layer 153 between the substrate 110 and the insulating layer 140, and in contact with the substrate. The protection layers illustrated in FIG. 5 may be a first protection layer 151, and a second protection layer 153. The second protection layer 153 may include the same material as the first protection layer 151.

Material components of the first protection layer 151 and the substrate 110 are selected to facilitate a strong bond between the first protection layer 151 and the substrate 110. For example, the second protection layer may include a glass material (e.g., a glass frit). The second protection layer 153 including the same material as the first protection layer 151 may be coated on an upper surface of the substrate 110 and then the insulating layer 140 may be arranged on the second protection layer 160, and thus a bonding force between the insulating layer 140 and the second protection layer 160 may be increased because the insulating layer 140 may include a glass material like the second protection layer 153, except for a small difference in the amount and type of the glass material. Thus, sequential bonding of the insulating layer 140, the second protection layer 153, and the substrate 110 may result in a higher bonding force than direct bonding of the insulating layer 140 to the substrate 110.

FIG. 6 is a cross-sectional view of a composite filler structure 12 in which an electrode 130 is disposed on an upper surface of a filler layer 120, according to an example embodiment. Upon comparing FIGS. 1 and 6, the electrodes 130, the filler layer 120, the insulating layer 140, the substrate 110, and the protection layer 150 are sequentially arranged in the composite filler structure 12 of FIG. 6. The filler layer 120 and the insulating layer 140 may be disposed between the electrodes 130 and the substrate 110, and thus a space between the electrodes 130 and the substrate 110 may be increased. Thus, a possibility that a leakage current is generated between the electrodes 130 and the substrate 110 may be reduced.

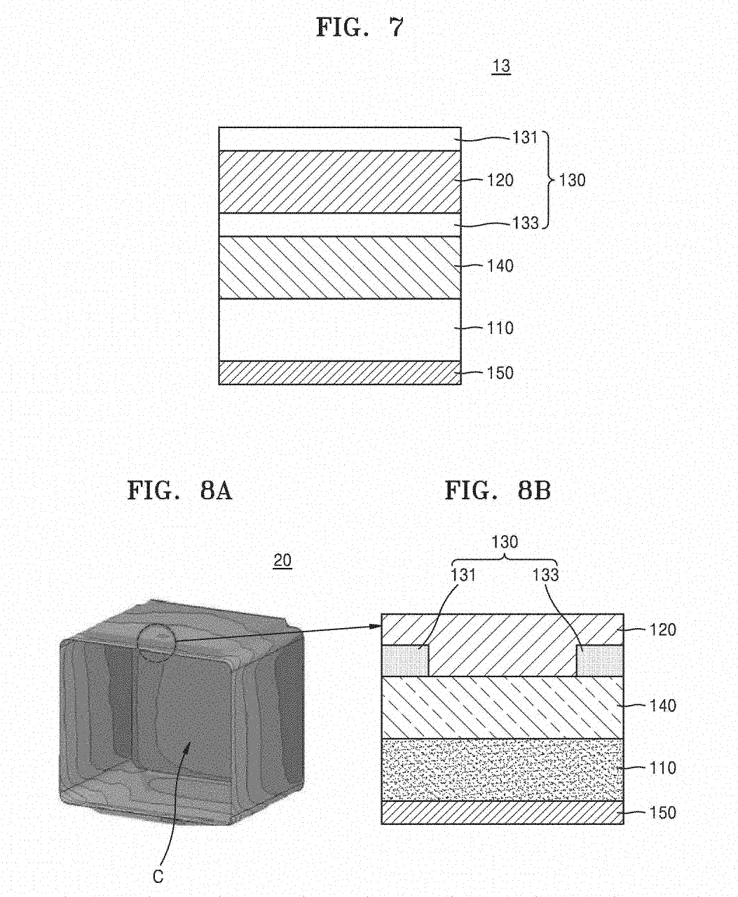

FIG. 7 is a cross-sectional view of a composite filler structure 13 in which first and second electrodes 131,133 are arranged with a filler layer therebetween, according to an example embodiment. Upon comparing FIGS. 1 and 7, the first electrode 131 of the composite filler structure 13 of FIG. 7 may be disposed on an upper surface of the filler layer 120, and the second electrode 133 may be disposed on a lower surface of the filler layer 120. The filler layer 120 of FIG. 7 may be a single layer. Alternatively, the filler layer 120 may include one or more openings defined therein. The openings may be filled with the same material as that of the insulating layer 140 so that the first electrode 131 and the second electrode 133 are spaced apart from each other.

FIG. 8A shows an electronic device 20 including a composite filler structure according to an example embodiment. FIG. 8B is a cross-sectional view of the composite filler structure present in the circled portion in FIG. 8A. As shown in FIG. 8A, the electronic device 20 may be an electric oven. The electronic device 20 may form a cavity C. The cavity C includes five surfaces, has a hexahedron shape, and inwardly opens forward. Thus, food that is to be heated may be placed in the cavity C. Although the electronic device 20 of FIGS. 8A and 8B includes the composite filler structure 10 of FIG. 1, the surfaces of the electronic device 20 may include the above-described composite filler structures 10, 11, 12, 13, or a combination thereof. For example, according to an example embodiment, an upper surface of the electronic device 20 may include each of the above-described composite filler structures 10, 11, 12, and 13. However, the present disclosure is not limited thereto. The above-described combination of composite filler structures 10, 11, 12, and 13 may be included in at least two surfaces of the electronic device 20, for example, upper and lower surfaces, both side surfaces, or a combination of an upper or lower surface and a side surface.

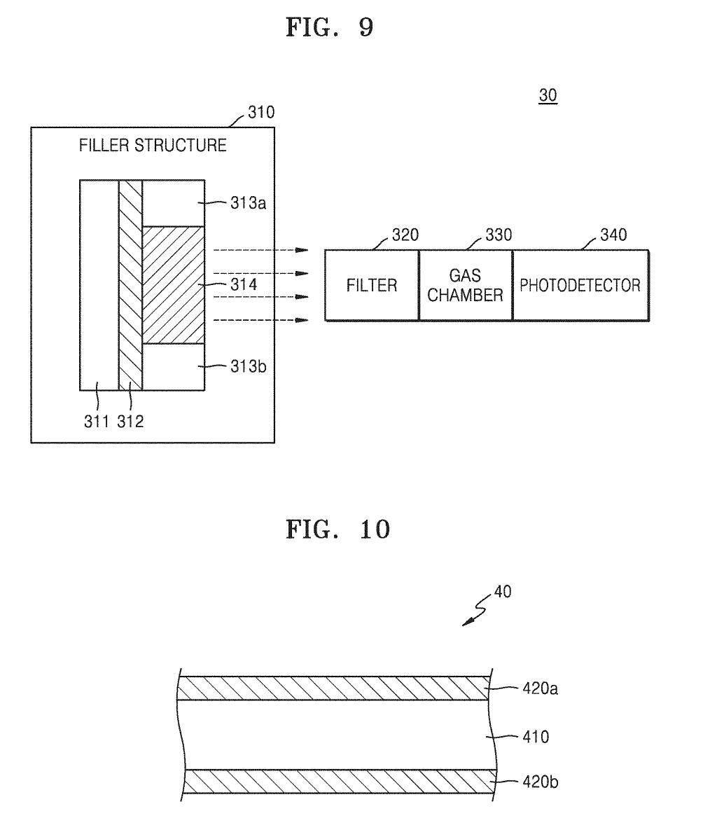

The composite filler structure according to an embodiment may be applied to a gas sensor which is a type of electronic device. FIG. 9 illustrates an example of a gas sensor 30 including a composite filler structure 310. The gas sensor 30 may be a gas sensor that detects the presence of gas using light. As shown in FIG. 9, the gas sensor 30 may include the composite filler structure 310, a filter 320, a gas chamber 330, and a photodetector 340.

The composite filler structure 310 may be configured to emit specific light such as infrared light while generating heat and may include a substrate 311, an insulating layer 312, electrodes 313a and 313b, and a filler layer 314,. The substrate 311 and the electrodes 313a and 313b shown in FIG. 9 may be formed of the same material as described for the substrate 110 and the electrode 130 shown in FIG. 1, but is not limited thereto. The substrate 311 and the electrodes 313a and 313b shown in FIG. 9 may be formed of a material more suitable for the gas sensor 30. For example, the substrate 311 may not be a conductive material. For example, the substrate 311 may include silica glass, quartz glass, polyimide, glass fiber, a ceramic, etc., and the electrodes 313a and 313b may be made of a silver-palladium (Ag—Pd) alloy, molybdenum (Mo), tungsten (W), platinum (Pt), an alloy thereof, and the like.

The insulating layer 312 may be formed of the same material as the insulating layer 140 described with reference to FIG. 1. For example, the insulating layer 312 may be formed of a material that is well adhered to adjacent layers, e.g., the substrate 311, the electrodes 313a and 313b, and the filler layer 314, and has excellent withstand voltage properties at a high temperature. The insulating layer 312 may include a glass frit and may not include an alkali oxide or may include a low amount of alkali oxide. For example, the insulating layer 312 may include an alkali oxide in an amount of 7 about wt % or less. The insulating layer 312 may have a glass transition temperature Tg of 400° C. or higher.

Also, the filler layer 314 may include a material that emits light, e.g., infrared light, by generating heat. For example, the filler layer 314 may include indium tin oxide (ITO), indium zinc oxide (IZO), zinc oxide (ZnO), tin oxide (SnO₂), antimony-doped tin oxide (ATO), Al-doped zinc oxide (AZO), gallium-doped zinc oxide (GZO), TiO₂, fluorine-doped tin oxide (FTO), or a combination thereof.

The filter 320 may selectively filter light emitted from the filler structure 310 such that light having a wavelength within a certain range passes from the filter 320 to the gas chamber 330. The gas chamber 330 may include a gas inlet (not shown) through which gas is introduced into the gas chamber from outside (e.g., external to the gas chamber and/or gas sensor) and a gas outlet (not shown) through which the gas is discharged. The gas chamber 330 may be formed of a material through which the light received from the filter 220 passes. The photodetector 340 may detect and the light passing through the gas chamber 130. The photodetector 340 may detect an amount of gas in the gas chamber 330 based upon the amount of detected light. The composite filler structure according to an embodiment may also be applied to the gas sensor 30 described above. The composite filler structure applied to the gas sensor may be heated in response to an electrical signal, but it is not limited thereto. Resistance of the composite filler structure applied to the gas sensor may change when contacted by particles, for example, gas particles, introduced from outside of the gas sensor. A magnitude of the electrical signal received by an electrode may vary due to the change in the resistance corresponding to the amount of the gas. A presence or an absence of gas, an amount of gas, and the like may be measured based on the received electrical signal.

In addition, the composite filler structure may be used in various applications requiring an insulation property, for example, in a defrosting heater of a refrigerator, a heat exchanger, a heating tool, tempered glass, a fuel cell, and a sealing material of a solar cell, etc.

In another embodiment, the composite filler structure disclosed above may be applied to a device that provides warmth to a user. For example, the composite filler structure may be applied to a hot pack, and may be included in clothing, such as a jacket or a vest, gloves, shoes, etc., that a user may wear on the body. The composite filler structure may be included inside the clothing or on an inner surface of the clothing.

In another embodiment, the composite filler structure may be applied to a wearable device. The composite filler structure may also be applied to outdoor equipment, which may be applied to a device that emits heat in cold environments.

On the other hand, the insulating layer including a low amount of alkali oxide, as described above, is not limited to a component of the composite filler structure. The insulating layer may also be applied to various devices to prevent insulation breakdown at a high temperature. The insulating layer according to an embodiment may be disposed on a functional layer that performs a specific function in response to an external signal, for example, an electrical or optical property inherent to an electrical signal. Here, the electrical property may represent a dielectric constant, a dielectric dissipation factor, a dielectric strength, a resistivity, an electrical conductivity, etc. The optical property may indicate a reflectance, a refractive index, etc. The filler layer described above may have high electrical conductivity as an inherent electrical property, and thus is an example of a functional layer in which heat is generated (e.g., emitted) in response to an electrical signal. Alternatively, the functional layer may be a heat absorbing layer, a refractive index changing layer, or a reflectance changing layer. That is, the insulating layer according to an embodiment may be applied to various types of devices by being disposed on the functional layer.

Alternatively, the insulating layer according to an embodiment may be applied to a substrate of an electronic device that is manufactured at a high temperature or is operable at a high temperature. FIG. 10 illustrates an example of a substrate 40 having an insulating property. A substrate formed of a material having a high mechanical strength is highly applicable to an electronic device. In general, a highly conductive metal material has a high mechanical strength. However, it is difficult to design a circuit board or the like on a metal substrate due to the electrical conductivity of the metal material. Thus, according to an embodiment, the substrate 40 may be provided with insulating properties by disposing insulating layers 420a and 420b on a base layer 410 including a material having a high mechanical strength and electrical conductivity.

As shown in FIG. 10, the substrate 40 having the insulating property may include the base layer 410 formed of an electrically conductive material and the insulating layers 420a and 420b disposed on and electrically insulating the base layer 410. The insulating layers 420a and 420b may be disposed on both sides of the base layer 410, for example, on an upper surface and a lower surface of the base layer, but are not limited thereto. The insulating layers 420a and 420b may be disposed on only some (e.g., not all) of the surfaces of the base layer 410.

The base layer 410 having the electrical conductivity may be the same material as previously described for the substrate 110, but is not limited thereto.

The insulating layers 420a and 420b may be formed of the same material as previously described for the insulating layer 140. For example, the insulating layers 420a and 420b may be formed of a material that is well adhered to the base layer 410 and has excellent withstand voltage properties at high temperatures. The insulating layers 420a and 420b may include a glass frit and may not include an alkali oxide or may include a low amount of an alkali oxide. For example, the insulating layers 420a and 420b may include an alkali oxide in an amount of about 7 wt % or less. The insulating layers 420a and 420b may have the glass transition temperature Tg of 400° C. or higher.

The substrate 40 having the above-described insulation property may be used as a substrate of a semiconductor device, a photoelectric conversion element, and a thin film solar cell, and may have, for example, a flat plate shape. A shape and a size of the substrate 40 may be appropriately determined according to the size of the semiconductor device, the light emitting device, the electronic circuit, the photoelectric conversion device, and the thin film solar cell to be used. When the substrate 40 is used for the thin film solar cell, the substrate 40 may have, for example, a rectangular shape with the longest sides having a length of greater than about 1 meter, or greater than about 2 m, or greater than about 3 m.

It should be understood that the example embodiments described herein should be considered in a descriptive sense only and not for purposes of limitation. Descriptions of features or aspects within each example embodiment should be considered as available for other similar features or aspects in other example embodiments.

While one or more example embodiments have been described with reference to the figures, it will be understood by those of ordinary skill in the art that various changes in form and details may be made therein without departing from the spirit and scope as defined by the following claims.

What is claimed is:

1. A composite filler structure comprising:
a substrate;
a filler layer spaced apart from the substrate and comprising a matrix material layer and a plurality of conductive filler particles;
an electrode in contact with the filler layer, and configured to provide an electrical signal to the filler layer; and
an insulating layer between the substrate and the electrode, the insulating layer comprising an alkali oxide in an amount of about 7 weight percent or less, based on a total weight of the composite filler structure.

2. The composite filler structure of claim 1, wherein the insulating layer, the matrix material layer, or a combination thereof comprises a glass frit.

3. The composite filler structure of claim 2, wherein the glass frit comprises an oxide glass frit.

4. The composite filler structure of claim 3, wherein the oxide glass frit comprises silicon oxide, lithium oxide, nickel oxide, cobalt oxide, boron oxide, potassium oxide, aluminum oxide, titanium oxide, manganese oxide, copper oxide, zirconium oxide, phosphorus oxide, zinc oxide, bismuth oxide, argon oxide, lead oxide, sodium oxide, or a combination thereof, and further comprises lithium, nickel, cobalt, boron, potassium, aluminum, titanium, manganese, copper, zirconium, phosphorus, zinc, bismuth, lead, sodium, or a combination thereof.

5. The composite filler structure of claim 2, wherein the insulating layer has a glass transition temperature of about 400° C. or greater.

6. The composite filler structure of claim 1, further comprising a protection layer in contact with the substrate and configured to prevent oxidation of the substrate.

7. The composite filler structure of claim 6, wherein the insulating layer comprises a material which is the same as a material in the protection layer.

8. The composite filler structure of claim 5, wherein an amount of the same material in the insulating layer and the protection layer is different from an amount in the protection layer.

9. The composite filler structure of claim 1, wherein a coefficient of thermal expansion of the insulating layer ranges from about 5 micrometers per meter per degree to about 12 micrometers per meter per degree.

10. The composite filler structure of claim 1, wherein the electrode is between the filler layer and the substrate.

11. The composite filler structure of claim 1, wherein the filler layer is between the electrode and the substrate.

12. The composite filler structure of claim 1, wherein the filler layer comprises a material configured to generate heat in response to an electrical signal.

13. The composite filler structure of claim 1, wherein the filler layer comprises a material having electrical resistance which changes when contacted with particles from outside of the composite filler structure.

14. The composite filler structure of claim 1, wherein the filler layer comprises carbon black, graphite, a metal, a conductive polymer, a metal powder, carbon nanotube, an oxide, a boride, a carbide, a chalcogenide, or a combination thereof.

15. An electronic device comprising,
a composite filler structure comprising:
a substrate;
a filler layer spaced apart from the substrate and comprising a matrix material layer and a plurality of conductive filler particles;
an electrode in contact with the filler layer, and configured to provide an electrical signal to the filler layer; and
an insulating layer between the substrate and the electrode, the insulating layer comprising an alkali oxide in an amount of about 7 weight percent or less, based on a total weight of the composite filler structure.

16. The electronic device of claim 15, wherein the insulating layer, the matrix material layer, or a combination thereof comprises a glass frit.

17. The electronic device of claim 15, wherein the substrate comprises a cavity defined therein.

18. The electronic device of claim 15, wherein the composite filler structure further comprises a protection layer in contact with the substrate and configured to prevent oxidation of the substrate.

19. The electronic device of claim 15, wherein an amount of alkali oxide in the insulating layer is less than an amount of alkali oxide in the protection layer.

20. The electronic device of claim 15, wherein the filler layer is configured to emit light by generating heat.

21. The electronic device of claim 20, further comprising:
a gas chamber comprising a gas inlet through which gas is introduced into the gas chamber; and
a photodetector configured to detect light transmitted by the filler layer and through the gas chamber.

22. An insulating substrate comprising:
a base layer comprising an electrically conductive material; and
an insulating layer disposed on the base layer, the insulating the comprising an alkali oxide in an amount of about 7 weight percent or less, based on a total weight of the insulating layer.
